(19) United States
(12) Patent Application Publication
(10) Pub. No.: US 2010/0221291 A1
(43) Pub. Date: Sep. 2, 2010

(54) COLLAGEN TUBES

(75) Inventors: Christian Gagnieu, Chassieu (FR); Vincent Guyot, Lyon (FR)

Correspondence Address: YOUNG & THOMPSON, 209 Madison Street, Suite 500, Alexandria, VA, 22314, US

(73) Assignee: ORTHOMED, SAINT JEANNET (FR)

(21) Appl. No.: 12/305,886

(22) PCT Filed: Jun. 7, 2007
(86) PCT No.: PCT/EP07/55614

§ 371 Date: Dec. 19, 2008

Related U.S. Application Data
(60) Provisional application No. 60/907,142, filed on Mar. 22, 2007.

(30) Foreign Application Priority Data
Jun. 22, 2006 (FR) 0605610

(51) Int. Cl. A61K 9/70; A61K 38/39; A61P 25/00; B32B 1/08; B05D 3/00

(52) U.S. Cl. 424/400; 514/12; 428/36.9; 428/36.91; 427/2.24

(57) ABSTRACT

The invention relates to collagen tubes, characterized in that they include a wall formed by a succession of continuous, cylindrical and coaxial collagen films. The invention also relates to tubules, bundles of tubules and combinations of tubes, tubules and bundles. These tubes are designed to be used in surgery, particularly in order to promote nerve regeneration.

The invention relates to tubes, bundles of tubes and combinations of tubes or bundles consisting of collagen, for use in biology and medicine as a growth medium and/or cell structure differentiation medium, especially in surgery, to stimulate the regrowth of transected nerves.

The invention also relates to a manufacturing process for continuous, cylindrical, coaxial collagen films, layered to form the walls of the said tubes.

Several techniques have been developed to repair nerve lesions, in particular transected nerves. One common technique is the surgical insertion of a tube linking the two ends of the transected nerve to guide the regrowth of the proximal section of the nerve towards the distal section and enable the two sections to join.

To create a tube that would stimulate nerve regrowth, numerous polymers were tested e.g. silicones, polyglycol acid or lactic acid polymers but none of these compounds meets the complex requirements of nerve regrowth. In particular, the tube should preferentially be made of an absorbable material.

Practitioners have defined mandatory criteria for the tubes, to allow for maximum regrowth of transected nerves, in particular:
The tubes must consist of a biocompatible material that is absorbable after the processes involved in the growth and maturing of the axons have been stimulated and completed.
The tubes must have enough mechanical resistance to withstand external pressures while remaining sufficiently flexible to avoid conflict with the nerve.
The exterior and interior of the tube must be smooth to avoid damaging the surrounding tissues or the ends of the nerve.
The wall of the tube must have low-level porosity, enabling the circulation of trophic factors but not the circulation of cells.
The tubes must be able to be sterilised.

Collagen is a material that is particularly suitable for surgical implants. It has a range of physical, chemical and biological properties that make it a first-line material for tubes complying with the characteristics listed above. Collagen is biocompatible and absorbable. Its absorption rate can be fine-tuned and it can combine with factors that stimulate nerve regrowth. Moreover, it has low-level antigenicity, a role in cell growth and cell differentiation as well as significant haemostatic power.

The collagen molecules are animal proteins located in the extracellular matrix. Their structure contains one or more triple-helix areas. A triple helix is obtained by combining three alpha chains, each consisting of 1,050 amino acids. At the end of the chains, non-helicoidal areas containing some forty amino acids (telopeptides) allow the collagen molecules to combine. The orderly arrangement of macro-molecules leads to the formation of fibres.

Collagen is extracted from source tissues by methods well known to those skilled in the art. There are several types of collagen with various levels of structure.
Collagen is described as “native” when its entire structure within the tissues (triple, helix and telopeptides) is retained on extraction.
Collagen can be split enzymatically or chemically in the telopeptides: it is then referred to as atelocollagen.
When the three alpha chains in the triple helix are separated by denaturing (e.g. heating), the collagen is said to be denatured.

Various types of collagen have been discovered. Some of them have been isolated and produced industrially. These are mainly types I, II and IV.

Depending on the type of collagen and the techniques used to prepare the collagen solution and manufacture the tube, the products will have different physical, chemical and metabolic characteristics.

There are numerous documents describing collagen tubes used to stimulate and guide nerve regrowth.

Patent EP 0 156 740 (U.S. Pat. No. 4,814,120) describes a method for the manufacture of collagen tubes that can be used in vascular prostheses and nerve sutures. These tubes consist of a single layer of collagen.

U.S. Pat. Nos. 4,963,146 and 5,026,381 describe a tube used for the regeneration of transected nerves, consisting of at least two layers of collagen I with different permeable qualities, including one external porous layer.

U.S. Pat. No. 6,090,117 refers to a tube that can regenerate a portion of missing nerve. The membrane of this tube is covered with two, interior and exterior, layers of gelatin or collagen. The tube stimulates the infiltration of capillary veins. The tube is filled with a collagen body consisting of cavities which are filled with a matrix-gel containing collagen, laminin, proteoglycans and growth factors.

U.S. Pat. No. 6,716,225 (application WO 2003/011149) describes a tubular matrix consisting of collagen. The internal diameter of the matrix lies between 0.1 and 10 mm. The external wall of the matrix is characterised by its roughness.

U.S. Pat. No. 6,953,482 describes a device used to stimulate nerve regeneration, consisting of a tubular-shaped support, a matrix in the form of a sponge filling the tube, and a tubular structure consisting of collagen fibres to guide the regrowth of the nerve.

US patent application 2004/0170664 describes a tube for use in nerve regeneration consisting of a absorbable, smooth, non-porous outer wall comprising a single collagen film folded back on itself, with the two ends sealed by bonding or adhesive. The tube can by filled with a collagen matrix.

U.S. Pat. No. 3,562,820 describes a succession of mucous layers built up using fibrous collagen adhesive. The layers are a non-homogeneous composite mixture.

The invention described in document WO82/03764 comprises several layers containing collagen combined with the living cells responsible for the collagen molecular arrangement.

Document EP 0943345 describes several layers of non-woven collagen formed randomly at the time of deep-freezing and vacuum dehydration.

U.S. Pat. No. 5,207,705 describes several layers consisting of a mixture of polyurethane foam to which collagen was added when the polyurethane became reticulated.

The tubes described in the standard documents for this technique have a number of disadvantages.

If the external membrane of the tube is too porous, cells such as fibroblasts can penetrate the tube due to scar invasion. This invasion creates an obstacle to nerve regrowth.

If the tubes have a rough outer surface, the roughness may damage the surrounding tissues. Likewise, the presence of areas of bonding or adhesive on the external membrane is a disadvantage because the asperities cause irritation.

Generally speaking, the presence of matrix in the tubes prevents the nerve from being effectively guided during the regrowth period.

Finally, because of the method of preparation, the tubes are, in most cases, very expensive.

This invention therefore proposes a new type of collagen tube for use in stimulating nerve regeneration. Tubules are welded together and inserted into the tube as a bundle to allow the groups of axons, which are components of the nerve, to be guided individually. Each tube and tubule consists of a succession of continuous, cylindrical, coaxial collagen films, preferentially with more or less the same level of porosity.

### DESCRIPTION OF THE INVENTION

This invention relates to a collagen tube characterised in that it includes an outer wall comprising a succession of continuous, cylindrical, coaxial collagen films.

The expression “continuous film” means that the film consists of a single length, with no areas of bonding or adhesive. The films are tube-shaped and are more or less cylindrical. The term “cylindrical tube” refers to all cylinders resulting from a directrix, preferentially a circular directrix. The adjective “coaxial” indicates that all the films assembled share the same axis viz. the axis of the tube. The films should preferentially be transparent, homogeneous and contain no, or very few, aggregates. The films should preferentially be non-porous i.e. their porosity should not be greater than 1 μm. Molecules with a molecular weight of approximately 70 kDa can be diffused through the wall of the tube. The average absorption time for the tube can be controlled and varied depending on the treatment.

Advantageously, each collagen film constituting the wall has a thickness of between 0.5 and 4 μm.

The invention refers more particularly to a collagen tube with a wall consisting of a succession of at least 5 continuous, cylindrical, coaxial collagen tubes. The wall preferentially consists of a succession of 5 to 30 films and, in particular, 10 to 15 films.

Those skilled in the art will know how to adapt the number of films in the wall of the tube depending on the properties required e.g. resistance to traction, flexibility or thickness. These characteristics are selected in accordance with the purpose of the tube

The films assembled to create the wall of a tube may all have the same composition. According to another aspect of the invention, the films constituting a given wall can have different compositions i.e. consist of different types or mixes of collagen. In particular, the films constituting the inner and outer layers may consist of different mixes of collagen.

The films constituting the walls can contain Type I collagen, Type III collagen or a combination of the two. The mix can be made of all the possible relative proportions of collagen of Types I and III. The preferred composition is a mixture comprising 85% to 100% Type I collagen and 0 to 15% Type III collagen.

According to another embodiment of the invention, the films can include Type IV collagen, either on its own or combined with other types of collagen, in all possible relative proportions. The preferred composition is a mixture of 1% to 100% Type IV collagen (by weight) and 0 to 99% Type I collagen (by weight), or a mixture of Type I+Type III collagen in the aforesaid proportions.

Advantageously, the films constituting the innermost section of the wall should consist of Type IV collagen, while the other, more external films should consist of Type I collagen or a mixture of Type I+Type III collagens.

The collagens used can come from several different species, in particular cattles or pigs.

According to a preferred embodiment, the collagen used is atelocollagen i.e. a collagen that has been subjected to cleavage in the telopeptides (Rousseau & Gagnieu, Biomaterials, 2002).

According to another aspect of the invention, the collagen constituting the tube walls is reticulated. Reticulation is achieved using traditional techniques known to those skilled in the art. For example, it can be achieved by radiation. Preferentially, the reticulation reaction will be achieved by soaking the collagen in a formaldehyde solution at a concentration of between 0.01 and 2% for a period of between 1 minute and 72 hours, at a pH of between 3 and 9.5. The parameters are adapted depending on the rate of reticulation required.

The collagen tube according to the invention can be used for a wide range of applications. It is particularly designed to be used in nerve surgery, to guide and protect a transected nerve during regrowth.

In this specific application, the section of the collagen tube is adapted to suit the section of the nerve that is to be guided during regrowth. Ideally, the internal diameter of the tube should be equal to the section of the nerve being regenerated.

Generally speaking, the internal diameter of the collagen tube according to the invention measures between 50 μm and 10 mm. Preferentially it measures between 1 and 7 mm.

The invention also relates to small-diameter tubes, herein below called “tubules” with an internal diameter equal to, or less than, 500 μm and preferentially between 50 and 200 μm. The optimum diameter is 100 μm.

The invention also refers to a bundle of collagen tubes characterised in that they include at least two tubes according to the invention, aligned in parallel to each other and joined to each other by bonding.

Preferentially, the tubes in the bundle should all be of the same length and have the same internal diameter.

According to a preferred aspect of the invention, the tubes in the bundle should have an internal diameter of less than 500 μm and more preferentially an internal diameter of between 50 μm and 200 μm. They are then known as “tubules”.

According to a preferred embodiment of the invention, the bonding of the tubes or tubules is achieved by partial fusion of the external membranes of the said tubes. Those skilled in the art will know how to determine the optimum parameters for the fusion reaction, to obtain partial fusion of the external membranes.

The bundle may comprise a number of tubes or tubules ranging from 2 to 60 and, in particular, comprise 2, 3, 4, 5, 10, 15, 20, 30, 40, 50 or 60 tubules, depending on the required diameter of the bundle.

The bundle is also characterised in that it is enveloped in a collagen sheath with a thickness of between 1 μm and 3 μm. By coating the bundle, it is given a smooth, uniform surface, making it easier to insert in a tube where necessary. The sheath should preferentially consist mainly of Type I collagen.

The bundle is advantageously subjected to a reticulation reaction after assembling the tubes, bonding them to join them then sheathing them with collagen film. Reticulation can be achieved using any of the conventional techniques known to those skilled in the art and, in particular, by using a formaldehyde solution as described above.

This invention also relates to a combination of tubes characterised in that it includes a tube in accordance with the invention, wherein at least one bundle of tubes in accordance with the invention is inserted longitudinally.

The respective diameters of the bundle and tube are adapted so that they can be combined. The bundle (resp. bundles) is (resp. are) inserted in the tube when dry.

The tubes and tubules according to the invention may be of any length. In general, a tube of an appropriate length is prepared and is then cut to the exact length required using conventional techniques known to those skilled in the art.

If the combination according to the invention is designed to be used for nerve regeneration, the length of the tube and bundle must be adapted to the length of the missing section of nerve.

In general, transected nerves lack a section of the order of several centimetres in length, up to as much as 15 cm. As far as the collateral nerves of fingers are concerned, the length of nerve missing ranges from 2.5 to 3 cm. For the reconstruction of larger nerves such as the median nerve, or the repair of damage to the brachial plexus, the missing nerve can have a length of 10 to 15 cm.

Preferentially, the length of the tube will be between 5 mm and 10 cm.

To ensure that the tube or combination of tubes according to the invention provide effective protection for the area of regeneration and the two ends of the nerve requiring repair, the tube should be longer than the area requiring regeneration. Ideally, the length of the tube should be greater than the length of the missing section of nerve to allow, in particular, for the suturing of the transected nerve ends in the tube. The additional length measures advantageously approximately 6 mm, leaving 3 mm on each side for suturing and providing good protection for the nerve ends.

According to a preferred aspect of the invention, the tube/bundle combination according to the invention is characterised in that the length of the tube is greater than the length of the bundle. In particular, the bundle will be of the same length as the missing section of nerve and the tube will be longer in length to ensure protection for the suture and ends of the nerves, preferentially 6 mm longer i.e. 3 mm on each side of the area requiring regeneration.

When surgically positioning the combination according to the invention on a level with the missing section of nerve, the tube containing the bundles of tubules should be sutured to the ends of the nerve by the surgeon. Whatever the nerve requiring reconstruction, it should first be dissected using the naked eye before using a microscope. The ends are recut to produce a “healthy” area on the two ends. The guide is then put in place using several microscopic sutures. The proximal and distal sutures may be “reinforced” using organic glue.

This invention also relates to a manufacturing process for collagen tubes as defined above wherein the collagen is rendered soluble in an appropriate solvent. The resultant solution is then deposited on a cylindrical support and dried. These two stages are repeated to obtain several layers of concentric, coaxial collagen films.

Those skilled in the art will select the most appropriate solvent to dissolve the collagen, to obtain a collagen film that is as homogeneous as possible and, especially, without any or with only very few aggregates. To avoid the formation of aggregates, the collagen solution must dry after being poured i.e. the solvent must evaporate as quickly as possible.

The aqueous solvents traditionally used to dissolve collagen evaporate fairly slowly, encouraging the formation of aggregates. When collagen is dissolved in the appropriate organic solvent, on the other hand, the solvent evaporates at a rate sufficient to avoid the formation of aggregates.

According to a preferred method of use of the invention method, the solvent should be polar in type. Advantageously, the solvent contains glycerol. The dissolution of collagen in this solution requires stirring for several hours.

Advantageously, the solvent contains methanol and is preferentially a mixture of methanol and water (methanol 30-100%, water 0-70% in volume) or pure methanol (98-100%). All forms of collagen can be used i.e. acid-soluble collagen, fibrous collagen, atelocollagen or denatured collagen but atelocollagen is preferred, at a high level of purity (approximately 99%).

This method has the advantage of a 5-minute drying time for a tube with an internal diameter of 1.5 mm. The time may increase depending on the diameter of the tube but the increase in drying time will be of the order of one minute. For an equivalent method using an aqueous solution, drying time would be greater than one hour.

To produce tubules, the diluted collagen solution advantageously contains a surfactant compound which is eliminated by later washing of the tubule. Preferentially, the dissolved collagen solution should not contain any aggregates greater than 50 μm. This is ensured by extrusion through a filter.

According to a preferred method of use of the method, the cylindrical support is a synthetic polymer tube with a smooth surface and a diameter equal to the internal diameter required for the collagen tube. Preferentially, the support will be made of PTFE.

The dissolved collagen solution is deposited by immersing the support in the solution. Between each immersion, the tube is dried in a dust free airflow. The immersion/drying stages are repeated as many times as there are films in the assembly. In particular, the stages will be repeated between 5 and 30 times to obtain a tube with a wall consisting of 5 to 30 layers of film.

The collagen in the tubes will then preferentially be subjected to a reticulation stage. The cylindrical support will not be removed until after the reticulation stage.

Preferably, the collagen tube formed by the succession of immersion/drying stages is then immersed in a pH-controlled formaldehyde solution then in water, then in a glycine solution followed by immersion in water and, finally, immersed in acetone before removing the moulds for the tubes.

The invention also refers to any collagen film likely to be produced in accordance with the method described above. Advantageously, the film is characterised in that it is transparent, is perfectly smooth and contains no, or few, aggregates.

To prepare a bundle of tubules, the tubules, still containing the support, are assembled longitudinally. The resultant bundle of tubules is plunged into an appropriate solvent to inflate and partially dissolve the outermost layers of each tubule. Once processed in this way, the bundle is then subjected to low longitudinal force to move the tubules closer together and the fusion of layers during solubilisation. The fast drying of the bundle under an air flow ensures that the fused areas are solid i.e. the tubules have bonded together.

After drying, the bundle is enclosed in a collagen film then subjected to a collagen reticulation stage. The moulds are removed after reticulation.

Advantageously, the reticulation of the collagen is achieved as follows: the bundle is immersed in a pH-controlled formaldehyde solution as described above then successively immersed in water, 0.1M glycine, water and acetone.

This invention relates to a collagen tube or a bundle or combination of collagen tubes in accordance with the invention, for use in surgery.

The invention also refers to the use of a tube or bundle or combination of tubes to prepare a surgical device designed to stimulate and guide the regrowth of a transected nerve.

The tubes according to the invention are particularly suitable for neurosurgery because they can be used to prepare a surgical device that stimulates and guides the regrowth of a transected nerve.

The collagen tube or the combination of tubes according to the invention can be used as “guides” enabling two ends of a transected or damaged nerve to rejoin and reconnect in any way whatsoever. This technique can be applied to motor nerves and sensory fibres.

### EXAMPLES

### Manufacture of the Tube Surrounding the Bundles of Tubules

A collagen solution is prepared by stirring and dissolving 0.25 g collagen in 50 ml glycerol-containing methanol at a concentration corresponding to 50-100% of the final collagen concentration. The solution obtained is homogenised by successive extrusions through sieves with 150 μm and 50 μm mesh.

A cylindrical, rectilinear PTFE mould with a diameter of between 500 μm and 10 mm is immersed in the collagen solution and withdrawn after 2-15 seconds at a speed of between 0.5 and 2 cm per second. The mould coated in the collagen solution is then subjected to rotation within a filtered air flow for 2 to 15 minutes to ensure that the solvent evaporates uniformly. The immersion and drying stages are carried out 2 to 30 times depending on the number of layers of collagen required. After the last immersion, the final drying is carried out for 15 minutes.

Collagen reticulation is obtained by immersing the mould supporting the layers of collagen in a 0.01-0.5% formaldehyde bath for a time ranging from 5 minutes to 24 hours. The mould is then immersed successively for 5 minutes in a water bath, 5-60 minutes in a 0.1M glycine bath, 30 minutes in a water bath then 5-15 minutes in acetone. Once the acetone has evaporated for 5-15 minutes in a filtered air flow, the PTFE mould is withdrawn, producing a multilayer reticulated collagen tube that is placed in a drying chamber for at least 16 hours until the humidity level is equal to, or less than, 15%. This method manufactures tubes of between 3 and 25 cm.

### Manufacture of Tubules

The collagen solution is prepared as above but it also contains 0.5% (v/v) of a surfactant product such as Tween 20. The solution is filtered three times over a 50 μm mesh. The PTFE moulds with a diameter of 50-200 μm are immersed for 1-5 seconds in this solution and withdrawn at a rate of between 3 and 10 centimetres per second. The solvent is evaporated by exposing the collagen-coated mould to an air flow for 5 minutes. This stage can be repeated 2 to 30 times.

### Manufacture of Tubule Bundles

The tubules supported by the PTFE moulds are gathered into bundles of 3 to 60 parallel units and the bundles are tied at the ends. The bundles are immersed in a methanol bath for 2 to 20 seconds without traction. They are then subjected to slight longitudinal traction to ensure that the tubules are in close contact with each other. This stage is followed by 5 to 15 minutes of drying under an air flow. The bundle is coated with collagen by undertaking 1-5 immersion/drying cycles in a methanol-containing collagen solution with a 0.1-0.5% concentration and containing 0.25% glycerol.

The bundles are reticulated, removed from the mould and dried according to the method described for the manufacture of tubes/external sheaths. This method produces bundles of 3 to 60 tubules.

### Combination of Tubes and Bundles of Tubules

The assembly of these two elements is carried out when they are dry. The diameter of the tube/sheath is greater than the diameter of the bundle and this in turn depends on the number and diameter of the tubules it contains. The bundle is inserted longitudinally into the sheath. Its length can be equal to, or less than, the length of the sheath.

### DESCRIPTION OF FIGURES

FIG. 1: Combination of a tube with a diameter of 500 μm and a bundle of 2×200 μm diameter tubes. Observation under a scanning electron microscope.

FIG. 2: Bundle of 4 tubes with a diameter of 200 μm. Observation under a scanning electron microscope.

FIG. 3: Combination of a tube with a diameter of 500 μm and a bundle of 10 tubes with diameters of 100 μm. Observation under a scanning electron microscope.

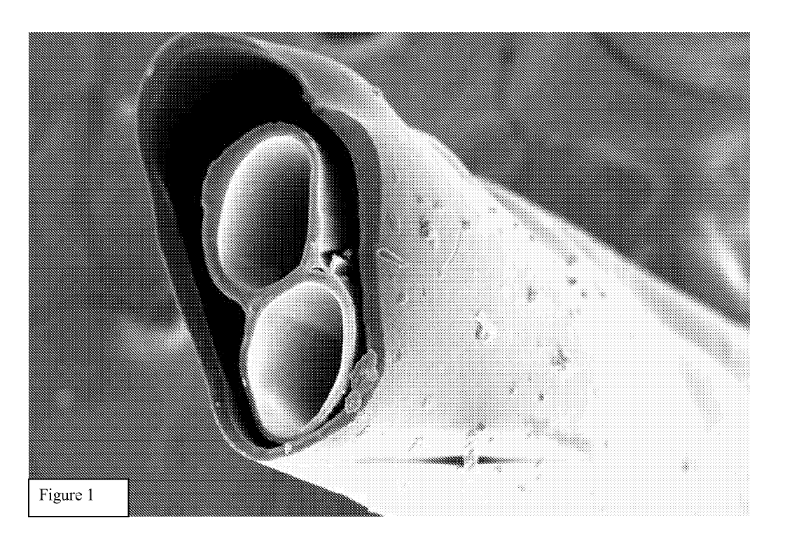

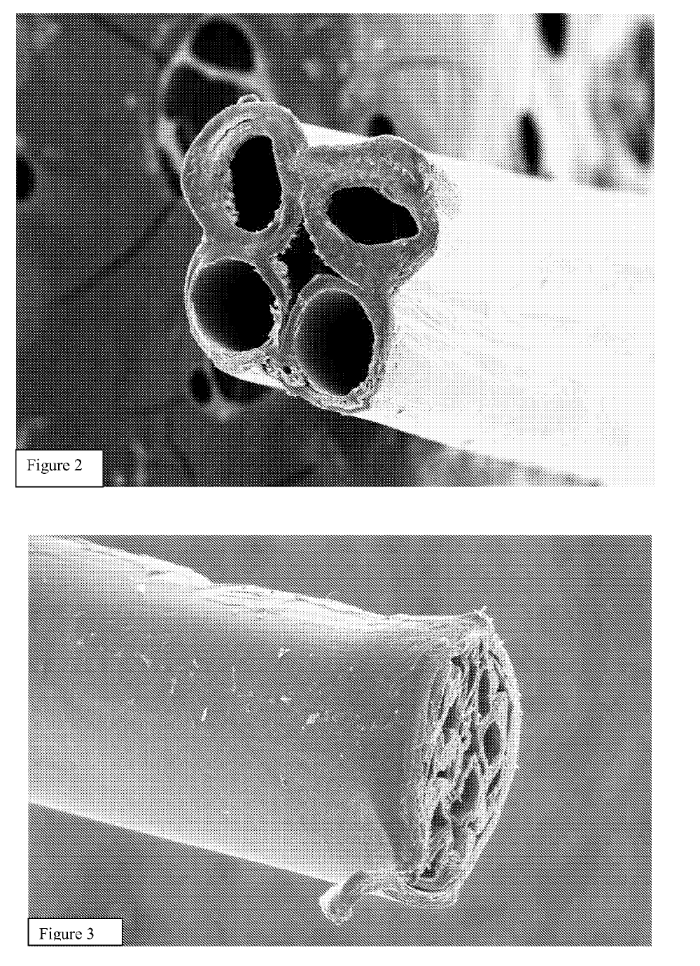

1. A collagen tube characterised in that it consists of a wall constituted of a succession of continuous, cylindrical, coaxial collagen films.

2. A tube according to claim 1, characterised in that each film constituting the wall has a thickness of between 0.5 and 4 μm.

3. A tube according to claim 1, characterised in that the wall is constituted by the layering of at least 5 films.

4. A tube according to claim 1, characterised in that the wall is constituted by the layering of 5 to 30 films, and preferably between 10 and 15 films.

5. A tube according to claim 1, characterised in that the films constituting the wall comprise Type I collagen, Type III collagen or a combination of both.

6. A tube according to claim 1, characterised in that the films constituting the inner layer of the wall comprise Type IV collagen.

7. A tube according to claim 1, characterised in that the collagen is atelocollagen.

8. A tube according to one of claim 1, characterised in that the collagen is reticulated.

9. A tube according to claim 1, characterised in that the internal diameter of the tube is between 50 μm and 10 mm.

10. A bundle of collagen tubes characterised in that it comprises at least two tubes according to claim 1, aligned in parallel and bonded to each other.

11. A bundle of tubes according to claim 10, characterised in that the tubes are tubules with an internal diameter of between 50 and 200 μm.

12. A bundle of tubes according to claim 10, characterised in that the bonding of the tubes is realized by partial fusion of the external membranes of the said tubes.

13. A bundle of tubes according to claim 10, characterised in that it is enveloped in a sheath consisting of collagen.

14. A combination of collagen tubes characterised in that it comprises one collagen tube that consists of a wall constituted of a succession of continuous, cylindrical, coaxial collagen films;
wherein at least one bundle of said tubes is inserted longitudinally according to claim 10.

15. A combination according to claim 14 characterised in that the length of the tube is between 5 mm and 10 cm in length.

16. A combination according to claim 15, characterised in that the length of the tube is greater than the length of the bundle of tubes.

17. A collagen tube manufacturing process according to claim 1, characterised in that the collagen is rendered soluble in an appropriate solvent, the resultant solution is deposited on a cylindrical support then dried under an air flow, these two stages are repeated to produce a succession of collagen films.

18. A method according to claim 17, characterised in that the solvent contains methanol.

19. A method according to claim 17, characterised in that the solvent is a mixture of methanol and water.

20. A method according to claim 17, characterised in that it comprises a collagen reticulation stage before the support is withdrawn.

21-22. (canceled)

23. A method of repairing nerve damage comprising steps of providing at least one said collagen tube of C1 and applying the at least one collagen tube to the damaged nerve.

24. A surgical device for use in stimulating and guiding regrowth in a transected nerve comprising at least one said collagen tube of C1.
